(19) United States
(12) Patent Application Publication
(10) Pub. No.: US 2024/0407649 A1
(43) Pub. Date: Dec. 12, 2024

(54) MEDICAL IMAGE DIAGNOSTIC APPARATUS AND OPERATION METHOD OF MEDICAL IMAGE DIAGNOSTIC APPARATUS

(71) Applicant: FUJIFILM Healthcare Corporation, Chiba (JP)

(72) Inventors: Takashi NISHIHARA, Chiba (JP); Wataru YOKOHAMA, Chiba (JP); Hisako NAGAO, Chiba (JP)

(73) Assignee: FUJIFILM Healthcare Corporation, Chiba (JP)

(21) Appl. No.: 18/731,361

(22) Filed: Jun. 3, 2024

(30) Foreign Application Priority Data
Jun. 9, 2023 (JP) 2023-095384

(51) Int. Cl. A61B 5/00; A61B 5/055

(52) U.S. Cl. CPC A61B 5/0033; A61B 5/055; A61B 5/742; A61B 5/7465

(57) ABSTRACT

Provided are a medical image diagnostic apparatus capable of appropriately instructing a subject to perform a requested motion in an examination, and an operation method of the medical image diagnostic apparatus.

A medical image diagnostic apparatus that captures a tomographic image of a subject includes: a first display unit that is visible to the subject under an examination; and a processor. The processor acquires first information including information on an examination to be executed, and displays, on the first display unit, second information including information on a motion to be performed by the subject, based on the first information.

### CROSS-REFERENCE TO RELATED APPLICATION

The present application claims priority under 35 U.S.C § 119 (a) to Japanese Patent Application No. 2023-095384 filed on Jun. 9, 2023, which is hereby expressly incorporated by reference, in its entirety, into the present application.

### BACKGROUND OF THE INVENTION

### 1. Field of the Invention

The present invention relates to a medical image diagnostic apparatus and an operation method of the medical image diagnostic apparatus, and particularly relates to a medical image diagnostic apparatus that captures a tomographic image of a subject in a bore, and an operation method of the medical image diagnostic apparatus.

### 2. Description of the Related Art

A medical image diagnostic apparatus that captures a tomographic image of a subject in a bore, such as a magnetic resonance imaging (MRI) apparatus or a computed tomography (CT) apparatus, is known. An examination using the medical image diagnostic apparatus includes an examination in which the subject is asked to perform a specific motion. For example, an MRI examination of an elbow includes an examination of imaging the elbow by bending the elbow at various angles. In the related art, in this type of examination, an operator verbally instructs the subject to perform a requested motion for the subject.

JP2016-30125A discloses a technique of providing a display unit that is visible to a subject during an examination, and displaying a sentence input by an operator on the display unit as a guide display.

### SUMMARY OF THE INVENTION

The requested motion for the subject varies depending on the examination, and it is difficult to appropriately instruct the subject about the content of the motion. In addition, there is also a problem in that inappropriate instruction confuses the subject.

The present invention has been made in view of such circumstances, and an object of the present invention is to provide a medical image diagnostic apparatus capable of appropriately instructing a subject to perform a requested motion in an examination, and an operation method of the medical image diagnostic apparatus.

(1) A medical image diagnostic apparatus that images a subject in a bore, the apparatus comprising: a first display unit that is visible to the subject under an examination; and a processor, in which the processor acquires first information including information on an examination target part of the subject, and displays, on the first display unit, second information including information for causing the subject to move the examination target part based on the first information.

(2) The medical image diagnostic apparatus according to (1), in which the information for causing the subject to move the examination target part is information on a motion of physically moving a specific part of a body.

(3) The medical image diagnostic apparatus according to (1) or (2), in which the first information further includes information on an examination purpose and information on a motion to be performed by the subject.

(4) The medical image diagnostic apparatus according to any one of (1) to (3), in which the second information includes information on the part of the body to be moved and information on a method of moving the part.

(5) The medical image diagnostic apparatus according to (4), in which the information on the method of moving the part includes information on a start position of the movement of the part and information on an end position of the movement of the part.

(6) The medical image diagnostic apparatus according to (4), in which, in a case where the specific part of the body is moved in stages, the information on the method of moving the part includes information on a position at which the part is stopped.

(7) The medical image diagnostic apparatus according to any one of (1) to (6), in which the second information includes information representing the part of the body to be moved and a method of moving the part in an illustration or an animation.

(8) The medical image diagnostic apparatus according to any one of (1) to (7), further comprising: a first detection unit that detects the motion of the subject in the bore, in which the processor determines whether or not a requested motion has been performed based on a detection result of the first detection unit, and notifies the subject of a determination result.

(9) The medical image diagnostic apparatus according to (8), in which the processor displays, on the first display unit, information on the motion of the subject in the bore together with the second information based on the detection result of the first detection unit.

(10) The medical image diagnostic apparatus according to any one of (1) to (9), further comprising: a first detection unit that detects the motion of the subject in the bore, in which the processor generates guide information for the subject to perform a requested motion based on a detection result of the first detection unit, and notifies the subject of the guide information.

(11) The medical image diagnostic apparatus according to (10), in which the processor displays, on the first display unit, information on the motion of the subject in the bore together with the second information based on the detection result of the first detection unit.

(12) The medical image diagnostic apparatus according to any one of (1) to (11), further comprising: a first detection unit that detects the motion of the subject in the bore; and a second display unit that is visible to an operator, in which the processor displays, on the second display unit, information on the motion of the subject in the bore based on a detection result of the first detection unit.

(13) The medical image diagnostic apparatus according to (12), in which the processor determines whether or not a requested motion has been performed based on the detection result of the first detection unit, and notifies the operator of a determination result.

(14) The medical image diagnostic apparatus according to (12) or (13), in which the processor generates guide information for the subject to perform a requested motion based on the detection result of the first detection unit, and notifies the operator of the guide information.

(15) The medical image diagnostic apparatus according to any one of (1) to (14), further comprising: a second detection unit that detects a specific motion by the subject, in which, in a case where the specific motion is detected by the second detection unit, the processor notifies an operator of the detected motion.

(16) An operation method of a medical image diagnostic apparatus that images a subject in a bore, the method comprising: acquiring first information including information on an examination target part of the subject; and displaying, on a first display unit that is visible to the subject under an examination, second information including information for causing the subject to move the examination target part based on the first information.

According to the present invention, it is possible to appropriately instruct the subject to perform a requested motion in the examination.

### BRIEF DESCRIPTION OF THE DRAWINGS

FIG. 1 shows a schematic configuration of an MRI apparatus.

FIG. 2 is a block diagram showing an example of a hardware configuration of an operation console.

FIG. 3 is a block diagram of main functions of the operation console.

FIG. 4 is a block diagram of main functions of a first display control unit.

FIG. 5 is a diagram showing an example of an input screen of examination information.

FIG. 6 is a diagram showing an example of a procedure of setting an option.

FIG. 7 is a conceptual diagram of information recorded in a display information database.

FIG. 8 is a diagram showing an example of display information.

FIG. 9 is a diagram showing another example of the display information.

FIG. 10 is a diagram showing still another example of the display information.

FIG. 11 is a flowchart showing a processing procedure of an examination using the MRI apparatus.

FIG. 12 is a functional block diagram of the MRI apparatus.

FIG. 13 is a block diagram of main functions of a motion detection unit, a determination unit, and a first display control unit.

FIG. 14 is a diagram showing an example of extraction of a feature point.

FIGS. 15A and 15B are diagrams showing an example of displaying a determination result.

FIGS. 16A and 16B are diagrams showing an example of displaying a motion state of a subject.

FIG. 17 is a diagram showing another example of displaying the motion state of the subject.

FIG. 18 is a diagram showing still another example of displaying the motion state of the subject.

FIG. 19 is a block diagram of functions of the operation console regarding a guide function.

FIG. 20 is a block diagram of main functions of the operation console.

FIG. 21 is a block diagram of main functions of the operation console.

FIG. 22 is a diagram showing an example of a specific motion and a content of an intention to be communicated.

### DESCRIPTION OF THE PREFERRED EMBODIMENTS

Hereinafter, preferred embodiments for carrying out the present invention will be described with reference to the accompanying drawings.

### First Embodiment

Here, a case where the present invention is applied to an MRI apparatus will be described as an example.

### MRI Apparatus

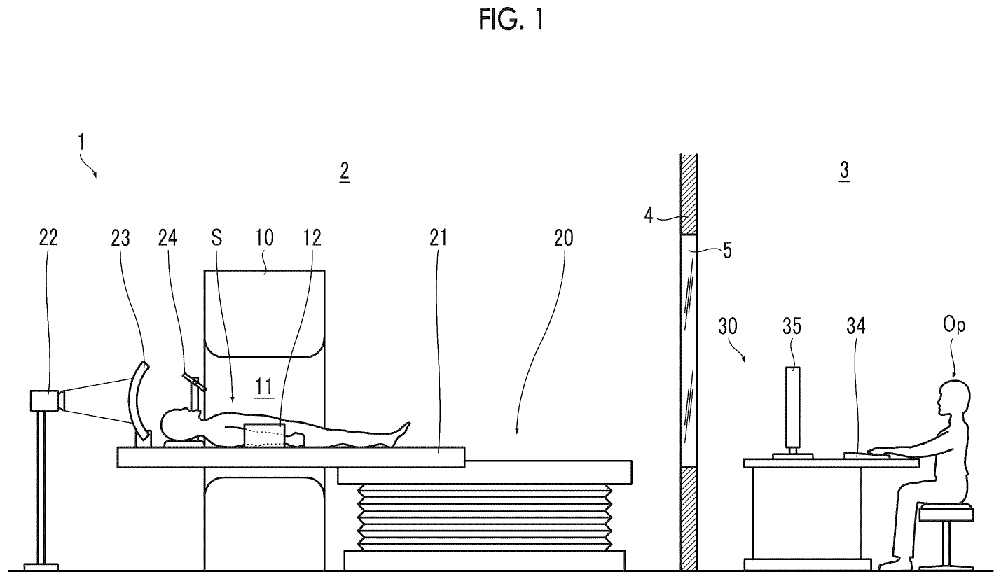

FIG. 1 is a diagram showing a schematic configuration of an MRI apparatus.

An MRI apparatus 1 shown in FIG. 1 includes a gantry 10, an examination table 20, an operation console 30, and the like. The gantry 10 and the examination table 20 are installed in an imaging room 2, and the operation console 30 is installed in an operation room 3. The imaging room 2 and the operation room 3 are separated by a partition wall 4. The partition wall 4 comprises a glass window 5. A state inside the imaging room 2 is observed from the operation room 3 via the glass window 5.

The gantry 10 has a bore 11 that constitutes an examination space (imaging space). As an example, in the present embodiment, the gantry 10 has a cylindrical bore 11 (has a so-called tunnel type structure). The gantry 10 can also adopt a so-called open type structure. A subject S is inserted into the bore 11 and is examined (imaged).

The gantry 10 has a static magnetic field magnet and a gradient magnetic field coil (not shown) in a housing. The static magnetic field magnet generates a static magnetic field in the bore 11. The static magnetic field magnet is composed of, for example, a permanent magnet, a superconducting magnet, or a normal conducting magnet. The gradient magnetic field coil is driven by a driving unit (not shown) to generate a gradient magnetic field.

The gantry 10 further has a reception coil 12. The reception coil 12 receives a magnetic resonance signal generated from the subject S. The reception coil 12 is disposed in an examination target part of the subject S. FIG. 1 shows an example of a case where the inspection target is an elbow. The magnetic resonance signal received by the reception coil 12 is reconstructed into an image and transmitted to the operation console 30.

The examination table 20 has a top plate 21 on which the subject S is placed. The top plate 21 is horizontally moved by being driven by a driving unit (not shown), and is inserted into the bore 11. In general, the subject S is placed on the top plate 21 in a supine posture and is inserted into the bore 11.

The top plate 21 comprises a screen 23 and a reflecting plate 24. An image is projected onto the screen 23 from a projection device 22 installed in the imaging room 2. The projection device 22 projects an image onto the screen 23 from a back side (so-called rear projection). The projection device 22 may be disposed outside the imaging room 2, and may project the image onto the screen 23 by using an optical fiber or the like. The reflecting plate 24 is disposed above a head of the subject S who is placed on the top plate 21 in a supine posture. The subject S observes the image (projection image) projected onto the screen 23 via the reflecting plate 24. Therefore, the image projected onto the screen 23 is composed of a mirror image whose left and right are reversed. A display unit composed of the projection device 22, the screen 23, and the reflecting plate 24 is an example of a first display unit that is visible to the subject S under the examination.

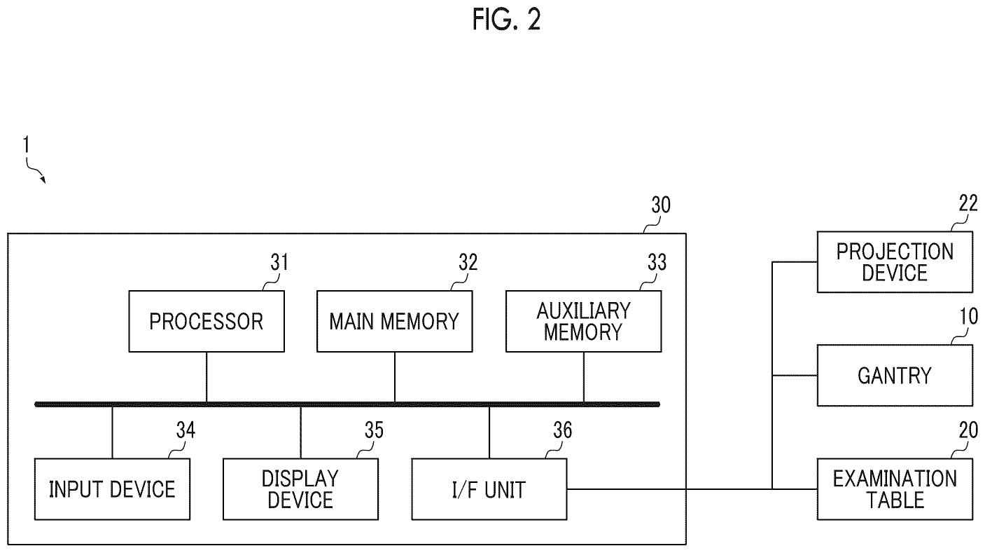

FIG. 2 is a block diagram showing an example of a hardware configuration of an operation console.

The operation console 30 receives an operation instruction from an operator Op, and operates the MRI apparatus 1 in accordance with the received instruction.

The operation console 30 has a configuration as a general computer. That is, the operation console 30 includes a processor 31, a main memory 32, an auxiliary memory 33, an input device 34, a display device 35, an interface unit 36, and the like.

As the processor 31, for example, a central processing unit (CPU) which is a general-purpose processor that executes a program and functions as various processing units is adopted. Various programs and data executed by the processor 31 are stored in the main memory 32 and/or the auxiliary memory 33. The program is synonymous with software.

The main memory 32 includes a random access memory (RAM) and a read only memory (ROM).

The auxiliary memory 33 is configured of, for example, a hard disk drive (HDD) or a solid state drive (SSD).

The input device 34 includes, for example, a keyboard, a mouse, a touch panel, and an operation panel.

The display device 35 is configured of, for example, a liquid crystal display (LCD) or an organic electro luminescence diode display (OLED display). The display device 35 is used for operating the MRI apparatus 1 and is also used for displaying various types of information, image viewing, and the like.

The interface unit 36 includes a network interface for connecting the operation console 30 to a network in addition to an interface for connecting the gantry 10, the examination table 20, and other peripheral devices.

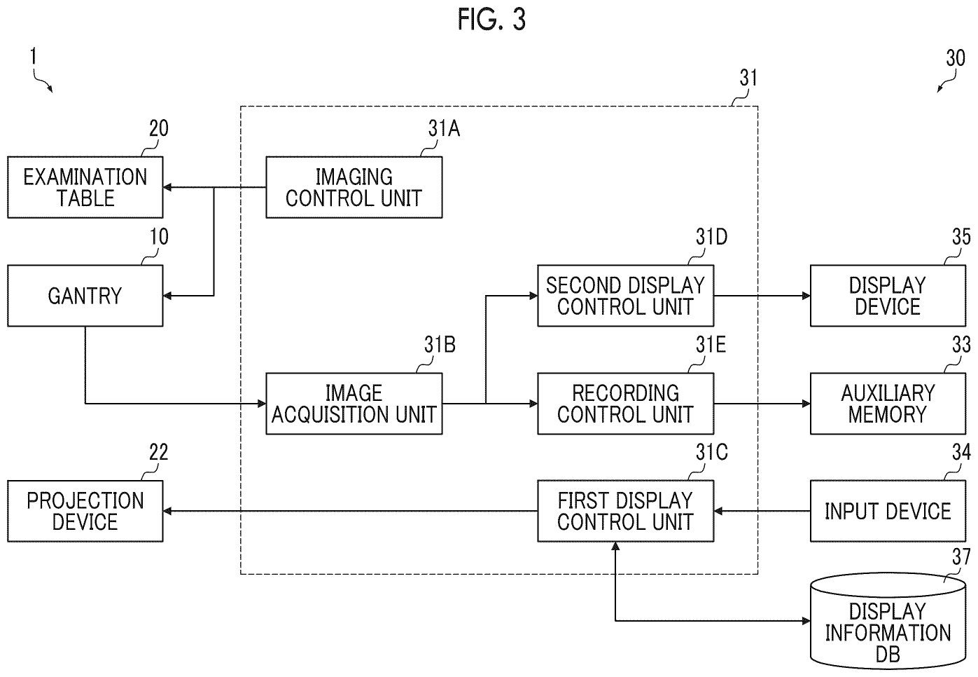

FIG. 3 is a block diagram of main functions of the operation console.

The operation console 30 mainly has functions of an imaging control unit 31A, an image acquisition unit 31B, a first display control unit 31C, a second display control unit 31D, a recording control unit 31E, and the like. The functions of the respective units are realized by the processor 31 executing a predetermined program.

The imaging control unit 31A controls the gantry 10 and the examination table 20 to control the imaging performed by the MRI apparatus 1.

The image acquisition unit 31B acquires the captured image from the gantry 10. The acquired image is an image reconstructed from the magnetic resonance signal, and is a tomographic image of the subject S.

The processing of the reconstruction may be performed by the operation console 30. In this case, the operation console 30 acquires the magnetic resonance signal from the gantry 10.

The operation console 30 performs predetermined signal processing on the acquired magnetic resonance signal to reconstruct an image.

The first display control unit 31C controls the display on the screen 23 performed by the projection device 22. As described above, the display of the screen 23 is observed by the subject S under the examination. In the present embodiment, in a case where the subject S is asked to perform a specific motion, information on the requested motion for the subject S is displayed on the screen 23. The first display control unit 31C controls the display. Details of the control will be described below.

The second display control unit 31D controls the display on the display device 35. The image (image generated by the image acquisition unit 31B) obtained by the imaging is displayed on the display device 35 under the control of the second display control unit 31D.

The recording control unit 31E controls the recording of data in the auxiliary memory 33. The image obtained by the imaging is recorded in the auxiliary memory 33 automatically or in response to an instruction of the recording from the operator Op under the control of the recording control unit 31E.

### Display Control on Screen

As described above, the display on the screen 23 by the projection device 22 is controlled by the first display control unit 31C. In a case where the examination in which the subject S is asked to perform a specific motion is performed, the first display control unit 31C displays the information on the requested motion for the subject S on the screen 23.

The requested motion for the subject S varies depending on the examination. The first display control unit 31C acquires information on the examination to be executed, and controls the display on the screen 23 so that information corresponding to the examination to be executed (information on the requested motion for the subject S in the examination) is displayed, based on the acquired information.

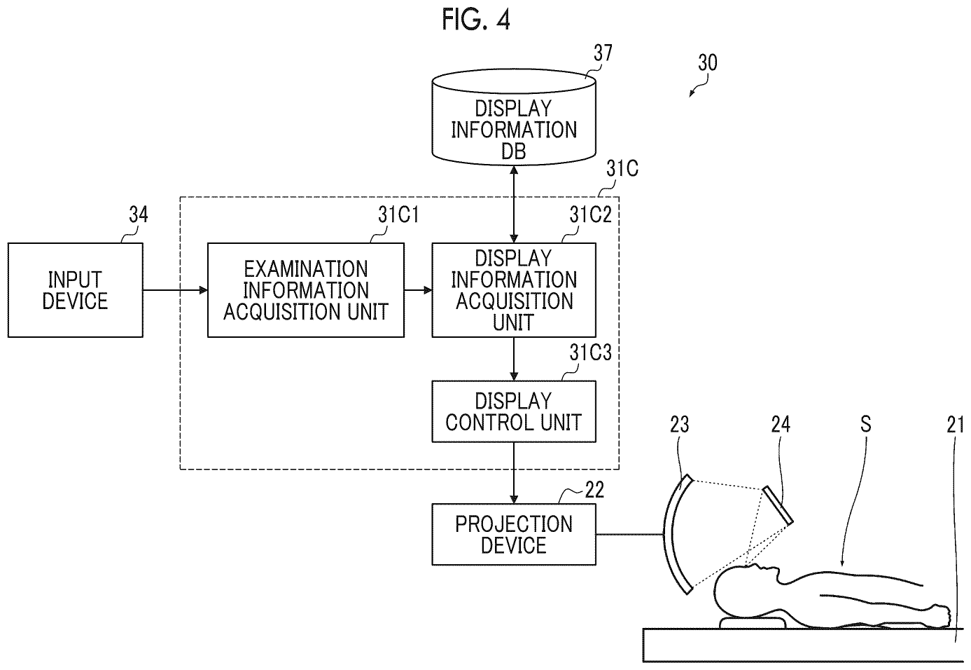

FIG. 4 is a block diagram of main functions of a first display control unit.

As shown in FIG. 4, the first display control unit 31C mainly has functions of an examination information acquisition unit 31C1, a display information acquisition unit 31C2, a display control unit 31C3, and the like.

The examination information acquisition unit 31C1 acquires information on the examination to be executed (examination information). In the present embodiment, the operator Op inputs the examination information via the input device 34. Therefore, the examination information acquisition unit 31C1 acquires the information input from the input device 34 as the examination information. The input of the examination information is performed, for example, by displaying a predetermined input screen on the display device 35.

The “examination information” includes at least “information on the examination target part”. In the present embodiment, information including “information on the examination target part”, “information on the examination purpose”, and “information on the motion to be performed by the subject” is acquired as the “examination information”. In the present embodiment, the examination information is an example of first information.

Here, the “information on the examination target part” is information on a part of a body that is a target of the examination (imaging). A head, a neck, an elbow, a shoulder, a hip joint, a knee, and the like fall under this category.

In addition, the “information on the examination purpose” is information about the purpose of the examination. A functional magnetic resonance imaging (fMRI) examination for brain function analysis, an optic nerve examination, a kinematic magnetic resonance imaging (kinematic MRI) examination for an orthopedic movable region imaging, and the like fall under this category.

In addition, the “information on the motion to be performed by the subject” is information on the motion to be performed by the subject under the examination in each examination. For example, in the kinematic MRI examination for the elbow, imaging is performed by bending the elbow in a predetermined direction and at a predetermined angle. In addition, for example, in the optic nerve examination, the imaging is performed by moving a viewpoint in a predetermined direction. As described above, the information on the motion determined for each examination is included in the examination information as the information on the motion to be performed by the subject. The content of the motion is defined as necessary. For example, for the motion of bending the elbow, the knee, or the like (motion such as bending and stretching), a bending direction, a bending angle, and the like are defined. In addition, for the motion of moving the viewpoint, a moving direction, a moving amount, and the like are defined.

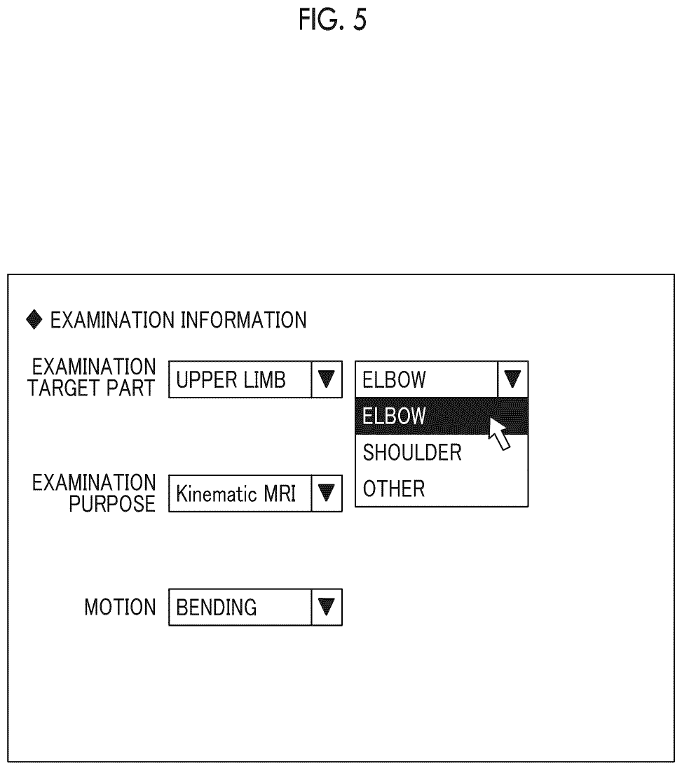

FIG. 5 is a diagram showing an example of an input screen of examination information. FIG. 5 shows an example of a case where the “information on the examination target part”, the “information on the examination purpose”, and “information on the motion to be performed by the subject” are input as the examination information.

As shown in FIG. 5, an input field of “examination target part” (an input field of the information on the examination target part), an input field of “examination purpose” (an input field of the information on the examination purpose), and an input field of “motion” (an input field of the information on the motion to be performed by the subject) are provided on a screen, and information according to the examination to be executed is input to each input field.

The input to each input field may be a text input method by the operator Op, or may be a method of displaying predetermined options and selecting and inputting one of the displayed options. FIG. 5 shows an example of a case where predetermined options are displayed in a pull-down menu, and one of the displayed options is selected to input necessary information.

In a case of displaying the options and inputting the necessary information, it is preferable that information on each item is input in a predetermined order, and the display content of the option of the next item is switched according to the previous selection result. For example, each information is input in the order of the “examination target part”, the “examination purpose”, and the “motion to be performed by the subject”, and the display content of the option is switched according to the previous selection result for the items of the “examination purpose” and the “motion to be performed by the subject”.

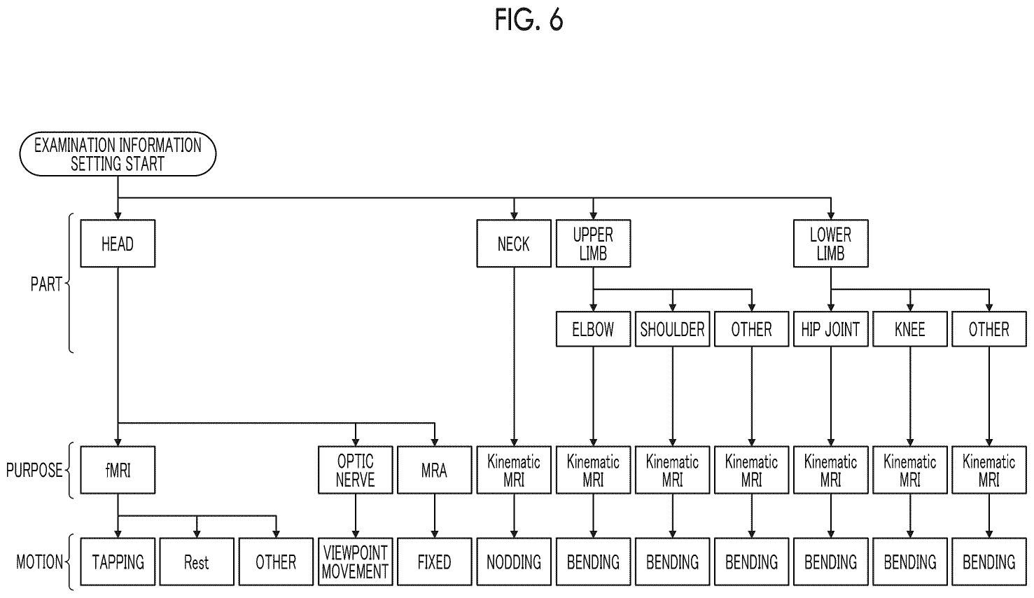

FIG. 6 is a diagram showing an example of a procedure of setting an option. FIG. 6 shows an example of a case where each information is input in the order of the “information on the examination target part”, the “information on the examination purpose”, and “information on the motion to be performed by the subject” in a case where the “information on the examination target part”, the “information on the examination purpose”, and “information on the motion to be performed by the subject” are input as the examination information.

First, the “examination target part” is selected. In the example shown in FIG. 6, “head”, “neck”, “upper limb”, and “lower limb” are displayed as the options of the examination target part. For the “upper limb” and the “lower limb”, more detailed parts are developed and displayed. Specifically, for the “upper limb”, “elbow”, “shoulder”, and “other” are developed and displayed as options of the detailed parts. In addition, for the “lower limb”, “hip joint”, “knee”, and “other” are developed and displayed as options of the detailed parts.

For the “examination purpose”, options to be displayed are determined according to the selection result of the “examination target part”. For example, in the example shown in FIG. 6, in a case where the “head” is selected as the “examination target part”, “fMRI” (functional MRI examination), “optic nerve” (optic nerve examination), and “MRA” ((magnetic resonance angiography) (MRA) examination) are displayed as the options of the examination purpose. In the example shown in FIG. 6, in a case where a part other than the “head” is selected as the examination target part, the examination purpose is automatically set to “kinematic MRI” (kinematic MRI examination).

In addition, for the “motion”, options to be displayed are determined according to the selection result of the “examination purpose”. For example, in the example shown in FIG. 6, in a case where the “head” is selected as the examination target part and the “fMRI” is selected as the examination purpose, “tapping”, “rest”, and “other” are displayed as the options of the motion. In the example shown in FIG. 6, the motion is automatically determined according to the selection results of the examination target part and the examination purpose, except for a case where the “head” is selected as the examination target part and the “fMRI” is selected as the examination purpose. For example, in a case where the “neck” is selected as the examination target part and the examination purpose is the “kinematic MRI”, the motion is automatically set to “nodding”.

Even though the “motion” is the same type of motion, even in a case where the contents thereof are different, the motion is treated as a different motion. For example, even though the same “bending” motion is performed, even in a case where a bending direction, a bending angle, and the like are different, the motion is treated as a different motion (different bending motion).

The display information acquisition unit 31C2 acquires the display information (information to be displayed on the screen 23) corresponding to the examination to be executed from a display information database (database: DB) 37 based on the examination information acquired by the examination information acquisition unit 31C1.

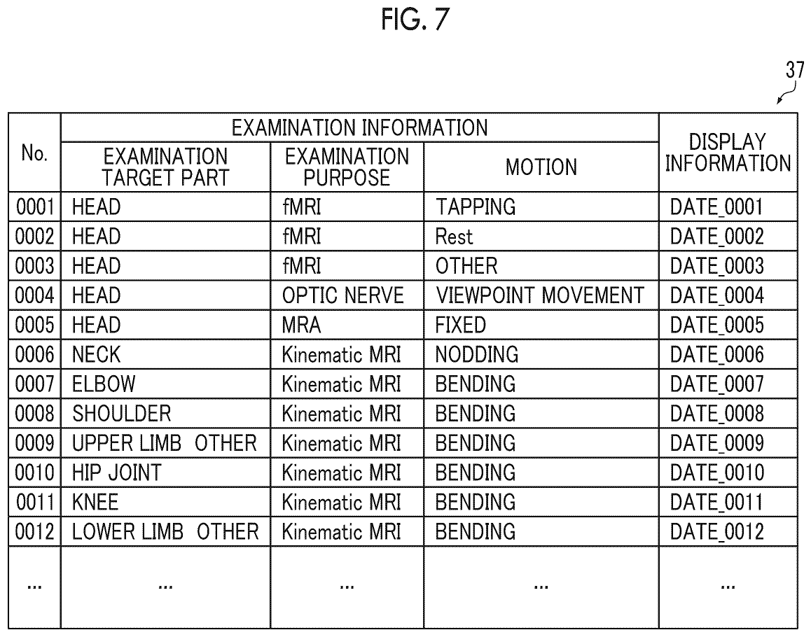

FIG. 7 is a conceptual diagram of information recorded in a display information database.

As shown in FIG. 7, the examination information and the display information are recorded in association with each other in the display information database 37. The display information database 37 is stored in, for example, the auxiliary memory 33.

In the present embodiment, the display information is composed of data of an image and/or text to be displayed on the screen 23. The image includes both a still image and a video. Details of the display information will be described below.

The display information acquisition unit 31C2 acquires the corresponding display information from the display information database 37 based on the examination information acquired by the examination information acquisition unit 31C1. For example, in a case where the examination information indicating that the examination target part is the “elbow”, the examination purpose is the “kinematic MRI”, and the motion is the “bending” is acquired, information of “DATE_0007” is acquired from the display information database 37 as the display information. As a result, the display information corresponding to the examination is acquired.

The display control unit 31C3 controls the projection device 22 to display the display information acquired by the display information acquisition unit 31C2 on the screen 23. As described above, in the present embodiment, the configuration is adopted in which the subject S observes the display of the screen 23 via the reflecting plate 24, so that the image displayed on the screen 23 is a mirror image in which the left and right are reversed.

As described above, in the present embodiment, the information on the examination to be executed (examination information) is acquired, and the display information corresponding to the acquired information is acquired from the display information database 37 and displayed on the screen 23.

### Display Information

The information displayed on the screen 23 (display information) as the information corresponding to the examination to be executed is information for causing the subject S to move the examination target part. In a case where the examination target part is caused to move by physically moving a specific part of the body, the display information includes information on the motion of physically moving the specific part of the body. In this case, the display information includes information on the part of the body to be moved and information on a method of moving the part. For example, in a case of the kinematic MRI examination of the elbow, the motion to be performed by the subject S is bending of the elbow. Therefore, in this case, information for issuing an instruction of the bending of the elbow (information indicating that the moving part is the “elbow” and the method of moving the part is “bending”) is displayed on the screen 23 as the display information. In the present embodiment, the display information is an example of second information.

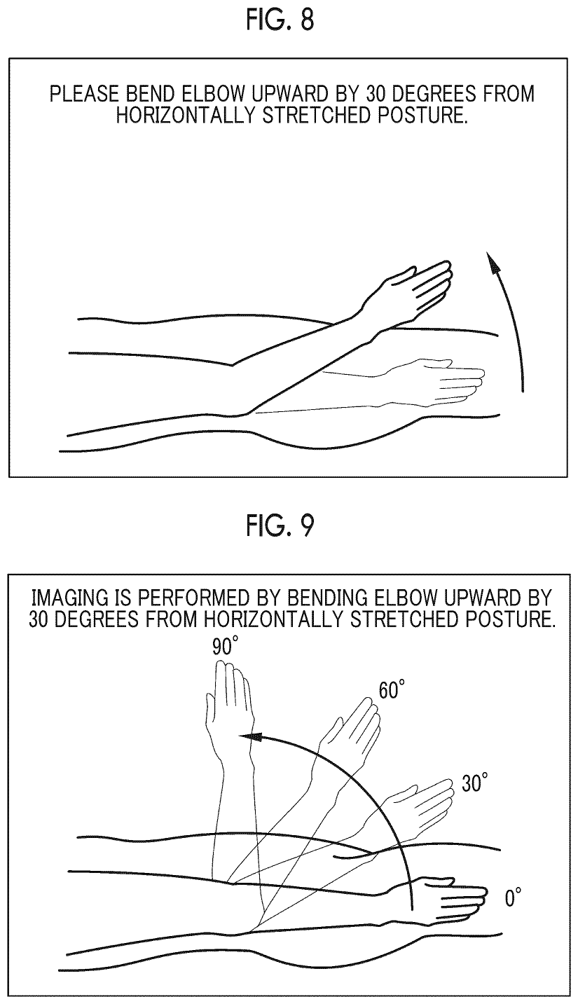

FIG. 8 is a diagram showing an example of the display information. FIG. 8 shows the display of the screen 23 observed via the reflecting plate 24. Therefore, the image actually displayed (projected onto) on the screen 23 is an image in which the left and right are reversed.

FIG. 8 shows an example of the display information in a case where the kinematic MRI examination of the elbow is performed. In particular, FIG. 8 shows an example of a case where the motion to be performed by the subject S is a motion of bending the elbow upward by 30 degrees from a horizontally stretched posture.

In addition, FIG. 8 shows an example of a case where the requested motion for the subject S is displayed in a text and an illustration.

As the text, a sentence for instructing the subject S to bend the elbow upward by 30 degrees from the horizontally stretched posture is displayed. As an example, a sentence (message) of “please bend elbow upward by 30 degrees from horizontally stretched posture” is displayed.

The illustration displayed shows the motion of bending the elbow upward by 30 degrees from the horizontally stretched posture. Instead of the illustration, the motion of bending the elbow upward by 30 degrees from the horizontally stretched posture may be displayed in an animation.

For the method of moving the part, it is preferable to specify a start position of the movement and an end position of the movement as shown in FIG. 8. In the example shown in FIG. 8, a position where the elbow is horizontally stretched is the start position of the movement, and a position where the elbow is bent upward by 30 degrees is the end position of the movement.

Here, the information on the elbow in FIG. 8 is an example of information on the part of the body to be moved. In addition, the text and the illustration shown in FIG. 8 are examples of information on the method of moving the part.

As described above, in a case where the requested motion for the subject S is a motion of a part of the body, it is preferable to show the requested motion for the subject S in an illustration or an animation. As a result, the subject S can easily understand the motion that himself/herself has to perform.

In a case where the examination target part is imaged a plurality of times by being moved in stages, the display information is switched for each imaging. The display may be switched automatically, or may be switched in response to an instruction from the operator Op. In a case of automatic switching, for example, a configuration can be adopted in which the display information is sequentially switched by using the imaging as a trigger.

FIG. 9 is a diagram showing another example of the display information.

FIG. 9 shows an example of display information in a case where the elbow is imaged a plurality of times by being bent in stages in the kinematic MRI examination of the elbow. FIG. 9 is an example of a case where the imaging is performed by bending the elbow upward by 30 degrees from the horizontally stretched posture. More specifically, FIG. 9 shows an example of a case where the imaging is performed in a state in which the elbow is horizontally stretched (0 degrees), in a state in which the elbow is bent by 30 degrees, in a state in which the elbow is bent by 60 degrees, and in a state in which the elbow is bent by 90 degrees.

As described above, in a case where a part of the body is imaged by moving the part of the body in stages, it is preferable to include information on each stop position in the display information. As a result, the subject S can easily understand a series of motions that himself/herself has to perform.

In the examination in which a part of the body is imaged by moving the part of the body in stages, an auxiliary tool may be used. For example, as in the above example, in the examination in which the elbow is imaged a plurality of times while being bent in stages, the elbow may be imaged using an auxiliary tool that can bend the elbow at a certain angle interval. In this case, information for issuing an instruction of the bending to the next angle may be displayed as the display information.

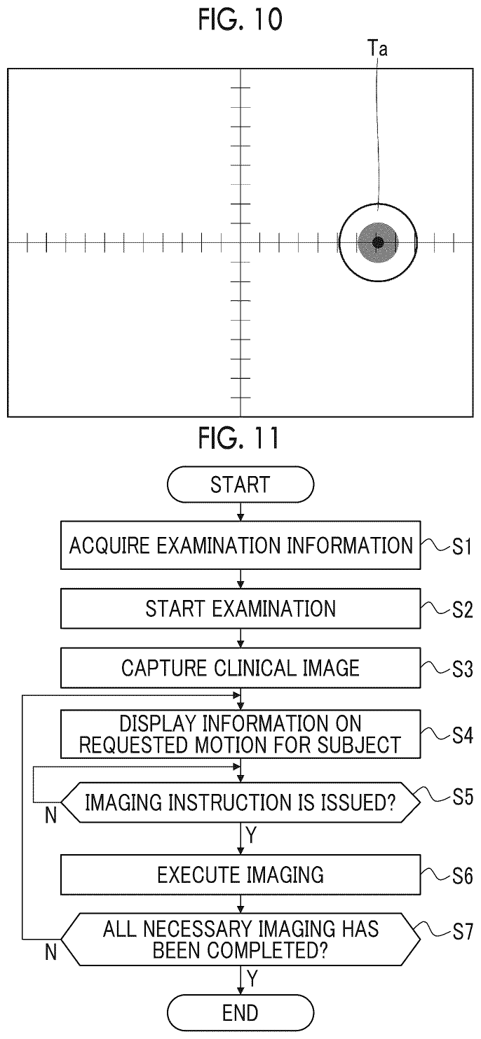

FIG. 10 is a diagram showing still another example of the display information.

FIG. 10 shows an example of display information in a case of performing the examination of the optic nerve. In particular, FIG. 10 shows an example of a case where the motion to be performed by the subject S is a viewpoint movement. In this case, as shown in FIG. 10, a target Ta is displayed on the screen 23. The target Ta is an image at which the subject S gazes in the examination. FIG. 10 shows an example of a case where the target Ta is configured with an image imitating an eye. In the examination, for example, the target Ta is moved on a preset route. The operator Op may move the target Ta. In this case, the operator Op moves the target Ta by using the display device 35 and the input device 34. The subject S performs a motion of following the target Ta with one eye or both eyes.

As described above, the screen 23 displays the information on the requested motion for the subject S in each examination as the display information.

In addition to the above, for example, in the functional MRI examination of the head, in a case where the subject S is asked to perform a tapping motion, an illustration or an animation of an index finger tapping is displayed. Similarly, in the functional MRI examination of the head, in a case where the subject S is asked to perform a motion of clasping or opening hands, an illustration or an animation of the motion of clasping or opening the hands is displayed. As in the present example, the subject S may be requested to move a part different from the examination target part depending on the examination purpose or the like. That is, the information for causing the subject S to move the examination target part includes information on a motion of physically moving a part of the body different from the examination target part.

In addition, for example, in the functional MRI examination of the head, in a case where the subject S is asked to perform a calculation operation, a calculation problem is displayed on the screen 23. For example, problems of performing calculation of subtracting 7 from 100 in sequence, such as “100−7=?” and “93−7=?”, are displayed. As described above, the motion to be performed by the subject S is not necessarily limited to the physical movement of a part of the body of the subject S. As with the calculation operation of the present example, an operation performed in the brain is also included. That is, the information for causing the subject S to move the examination target part includes information on the operation performed in the brain.

### Action of MRI Apparatus (Operation Method)

FIG. 11 is a flowchart showing a processing procedure of an examination using the MRI apparatus.

First, the examination information is acquired (step S1). As described above, in the present embodiment, the operator Op inputs necessary information from the input device 34, and the examination information is acquired.

In parallel with the acquisition of the examination information, information (for example, subject information) required in a general MRI examination is acquired.

After the acquisition of the examination information and the like, the subject S is set in the MRI apparatus 1. That is, the subject S is placed on the top plate 21. After the setting is completed, the examination is started (step S2).

In a case where the examination is started, first, a general clinical image such as a positioning image and a pre-measurement image is captured (step S3).

In a case where the capturing of the general clinical image is completed, the imaging corresponding to the examination content is started (step S3). At this stage, information on the requested motion for the subject S (information for causing the subject S to move the examination target part) is displayed on the screen 23 (step S4). The first display control unit 31C acquires the display information corresponding to the acquired examination information from the display information database 37, and controls the projection device 22 to display the information on the requested motion for the examination subject S on the screen 23. The subject S checks the display of the screen 23 and performs the requested motion.

The operator Op checks the motion of the subject S and issues an instruction to perform imaging. That is, in a case where the subject S performs the requested motion, an instruction is issued to perform imaging. The imaging control unit 31A determines whether or not the imaging instruction from the operator Op is issued (step S5). Then, the imaging is executed in response to the imaging instruction (step S6).

After the imaging, it is determined whether or not all the imaging necessary for the examination has been completed (step S7). In a case where it is determined that all the necessary imaging has been completed, the examination is ended.

As described above, with the MRI apparatus 1 according to the present embodiment, in a case of performing the examination in which the subject S is asked to perform a specific motion, the information on the requested motion is displayed on the screen 23. As a result, it is possible to appropriately instruct the subject S to perform the requested motion for the subject S in the examination.

### Modification Example

### (1) First Display Unit

In the above-described embodiment, the information on the motion to be performed by the subject S is projected onto the screen 23 by the projection device 22, and the subject S observes the information projected onto the screen 23 via the reflecting plate 24. The method of presenting, to the subject S, the information on the motion to be performed by the subject S is not limited to this. In addition, for example, the information on the motion to be performed by the subject S may be projected onto an inner wall surface of the bore 11, a wall surface of the imaging room 2, or the like by the projection device 22. In this case, the subject S directly observes an image projected onto the inner wall surface of the bore 11, the wall surface of the imaging room 2, or the like. In addition, the projection device 22 may directly project the information on the motion to be performed by the subject S onto the position of the reflecting plate 24 instead of projecting it onto the screen 23.

In addition, in the above-described embodiment, the configuration is adopted in which the projection device 22 is used, but a display device such as a liquid crystal display or an organic EL display may be used to present the information on the motion to be performed by the subject S to the subject S. In this case, the display device is disposed at a position where the subject S under the examination can visually recognize the display device. For example, a configuration can be adopted in which a display device whose position can be adjusted is provided in the gantry 10 to allow the subject S under the examination to visually recognize various types of information.

In addition, a configuration may be adopted in which the information on the motion to be performed by the subject S is output as a voice, in addition to the configuration in which the information is displayed on the screen 23 or the like, or instead of the configuration in which the information is displayed on the screen 23 or the like.

### (2) Examination Contents

In the above-described embodiment, as the examination performed using the MRI apparatus 1, a case of performing the functional MRI examination, the optic nerve examination, the kinematic MRI examination, or the like has been described as an example, but the application of the present invention is not limited to this. The present invention can be widely applied to the examination in which the subject S is asked to perform a specific motion.

### Second Embodiment

In the examination in which the subject is asked to perform a specific motion, it is preferable that the subject can check suitability of his/her executed motion. That is, it is preferable to be able to check whether or not his/her executed motion is a motion as requested. In the present embodiment, an MRI apparatus capable of checking suitability of his/her executed motion will be described.

### Device Configuration

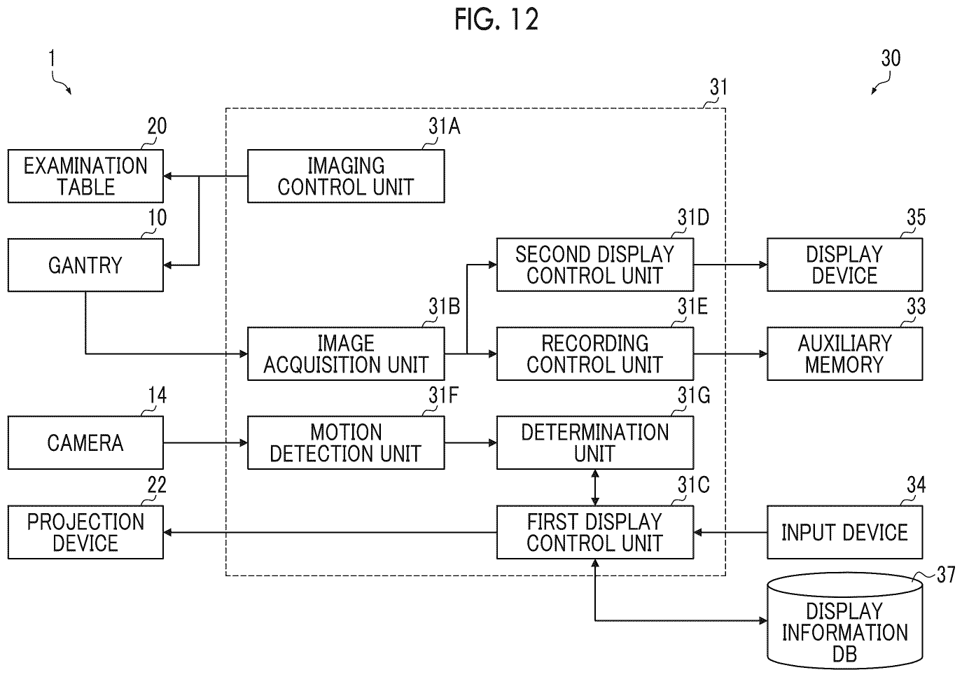

FIG. 12 is a functional block diagram of the MRI apparatus.

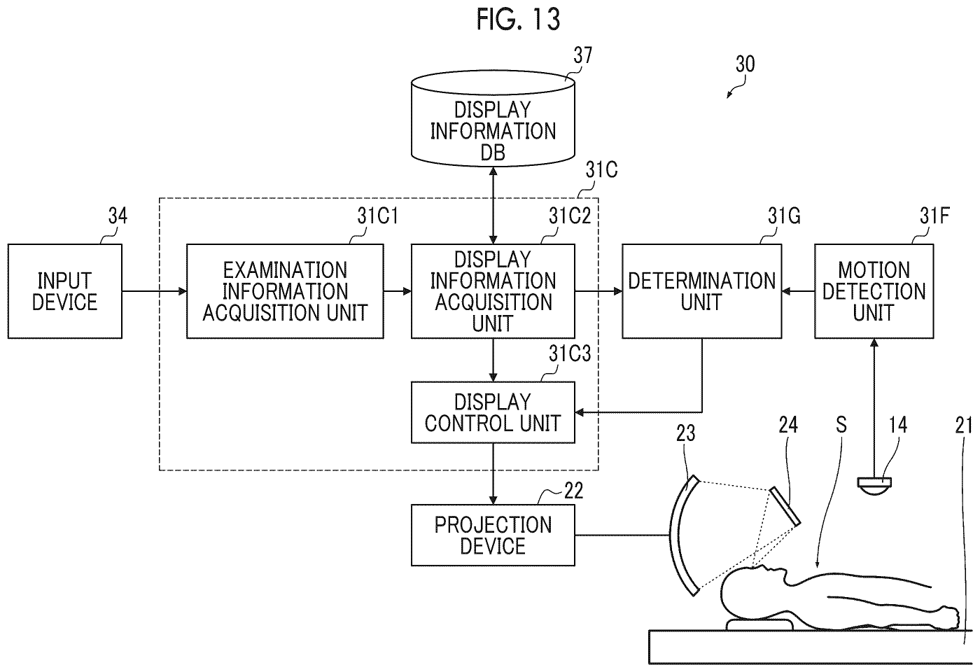

As shown in FIG. 12, the MRI apparatus 1 according to the present embodiment includes a camera 14. The camera 14 is a so-called digital camera, and electronically images the subject (the subject in the bore) S under the examination (see FIG. 13). An installation position of the camera 14 is not particularly limited. The camera 14 need only be installed at a position where the movement of the subject S in the bore can be checked from the captured image. Therefore, the camera 14 is not necessarily required to be provided in the gantry 10, but may be provided in the examination table 20 or the like. In addition, a plurality of the cameras 14 may be installed. By installing the plurality of cameras 14, it is possible to detect the motion of the subject S from various directions.

In addition, in the MRI apparatus 1 according to the present embodiment, the operation console 30 further has functions of a motion detection unit 31F and a determination unit 31G. The functions of the motion detection unit 31F and the determination unit 31G are realized by the processor 31 executing a predetermined program.

FIG. 13 is a block diagram of main functions of a motion detection unit, a determination unit, and a first display control unit.

The motion detection unit 31F acquires the image captured by the camera 14, and detects the movement of the subject S based on the acquired image. A known technique can be adopted for the process of detecting the movement from the image. For example, a method of extracting a feature point of a human body by image analysis and detecting the movement of the subject S from the movement of the extracted feature point can be adopted.

The feature point is extracted based on, for example, a skeleton model of the human body. Specifically, a joint portion is extracted as the feature point. For example, in a case where an examination target (imaging target) is the upper limb, the head, the neck, the right shoulder, the left shoulder, the right elbow, the left elbow, the right wrist, the left wrist, and the like are extracted as the feature points. In addition, in a case where the examination target is the lower limb, the right hip joint, the left hip joint, the right knee, the left knee, the right ankle, the left ankle, and the like are extracted as the feature points. In a case where the examination target is the head or the neck, the eyes, the nose, the ears, the mouth, and the like can be extracted as additional feature points.

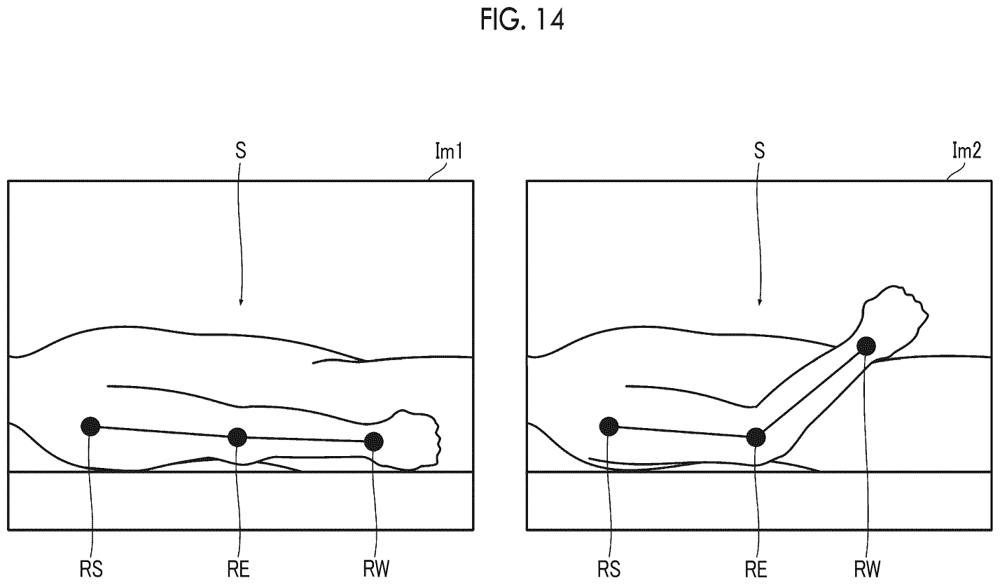

FIG. 14 is a diagram showing an example of extraction of the feature point. FIG. 14 shows an example of a case where the examination target is the right elbow. An image Im1 on a left side of FIG. 14 shows an example of an image captured in a state in which the elbow is horizontally stretched. In addition, an image Im2 on a right side shows an example of an image captured in a state in which the elbow is bent upward by about 60 degrees from the horizontally stretched posture. As shown in FIG. 14, the right shoulder, the right elbow, and the right wrist are extracted as the feature points from the image Im1 and the image Im2. In FIG. 14, reference numeral RS indicates a feature point of the right shoulder. In addition, reference numeral RE indicates a feature point of the right elbow. In addition, reference numeral RW indicates a feature point of the right wrist.

As shown in the image Im1 and the image Im2, the movement of the elbow of the subject S can be detected from the movements of the feature points RS, RE, and RW.

In the process of extracting the feature points from the image, for example, a trained model that has been trained through machine learning to extract the feature points from the image can be used.

In the present embodiment, the camera 14 and the motion detection unit 31F that detects the motion of the subject S from the image captured by the camera 14 are examples of a first detection unit.

The determination unit 31G determines whether or not the requested motion for the subject S has been performed based on the detection result of the motion detection unit 31F. The determination unit 31G acquires the information on the motion to be performed by the subject S from the display information acquisition unit 31C2. The display information acquisition unit 31C2 acquires the information on the motion performed by the examination subject S from the display information database 37 together with the display information. Therefore, the information on the motion performed by the subject S is recorded in the display information database 37 in association with the display information. The information to be recorded includes, for example, information on a part of the body to be moved and information on a method of moving the part. In addition, the information on the method of moving the part includes information on a start position of the movement and information on an end position of the movement. In a case where a specific part of the body is moved in stages, the information on the method of moving the part further includes information on a position at which the part is stopped. For example, in a case of the kinematic MRI examination of the elbow, in a case where the elbow is imaged at each position by being bent in stages, as the information on the motion (bending) to be performed by the subject S, (1) information on a direction of bending the elbow, (2) information on a start position (a start position of the movement), (3) information on a step (bending angle interval), (4) information on an end position (an end position of the movement), and the like are recorded. In addition, for example, in a case where the subject S is asked to perform a tapping motion in a case where the functional MRI examination of the head is performed, (1) information on duration (time for continuously performing the tapping motion), (2) information on an interval (time interval for executing the tapping motion), and the like are recorded. In addition, for example, in the kinematic MRI examination of the neck, in a case where the subject S is asked to perform a nodding motion, (1) information on a start position (a start position of the movement), (2) information on a step (a bending angle interval), (3) information on an end position (an end position of the movement), and the like are recorded.

The determination unit 31G determines whether or not the subject S has performed the requested motion based on the detection result of the motion detection unit 31F with reference to the information acquired from the display information acquisition unit 31C2. It is preferable to set an allowable range in the determination. For example, in a case where the subject S is asked to perform a motion of bending the elbow at a predetermined angle, an allowable range is set in a predetermined angle range before and after a target angle. In a case where the elbow is bent within an allowable range, it is determined that the requested motion has been performed. The allowable range is appropriately set according to the part of the examination target, the examination purpose, the motion content, and the like. In addition, a configuration may be adopted in which the operator Op can optionally set the allowable range.

The display control unit 31C3 displays the determination result of the determination unit 31G on the screen 23 to notify the subject S of the determination result.

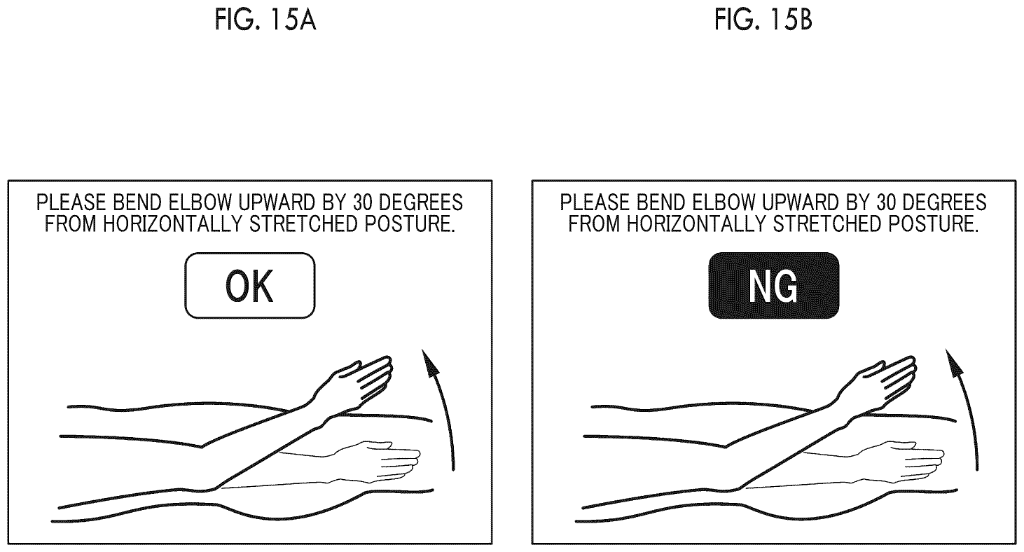

FIGS. 15A and 15B are diagrams showing an example of displaying the determination result.

As shown in FIGS. 15A and 15B, the determination result is displayed by being superimposed on the display information. FIG. 15A is a diagram showing an example of the display in a case where the requested motion has been performed. In this case, letters “OK” are displayed as the determination result. On the other hand, FIG. 15B is a diagram showing an example of the display in a case where the requested motion has not been performed. In this case, letters “NG” are displayed as the determination result.

The display control unit 31C3 controls the projection device 22 such that the determination result is displayed by being superimposed on the display information.

### Action

Here, processing from the detection of the motion of the subject S to the output of the determination result will be described.

In a case where the examination is started, the subject S under the examination is imaged by the camera 14. The imaging is performed at a predetermined frame rate. The time-series images captured by the camera 14 are added to the motion detection unit 31F.

The motion detection unit 31F extracts a feature point from the acquired image and detects the movement of the subject. The detection result is added to the determination unit 31G.

The determination unit 31G acquires the information on the motion to be performed by the subject S via the display information acquisition unit 31C2. The determination unit 31G determines whether or not the requested motion has been performed by the subject S based on the detection result of the motion detection unit 31F with reference to the acquired information. The determination result is added to the display control unit 31C3.

The display control unit 31C3 displays the determination result on the screen 23 by superimposing the determination result on the display information (see FIG. 14).

As described above, with the MRI apparatus 1 according to the present embodiment, the motion of the subject S is detected, and it is determined whether or not the motion as requested has been performed. Then, the subject S is notified of the determination result. As a result, it is possible to appropriately instruct the subject to perform the requested motion in the examination.

### Modification Example

### (1) Display of Motion State of Subject

Information on a motion state of the subject S detected by the motion detection unit 31F may be displayed together with the display information. As a result, the subject S can easily grasp his/her own state and can more easily execute the requested motion.

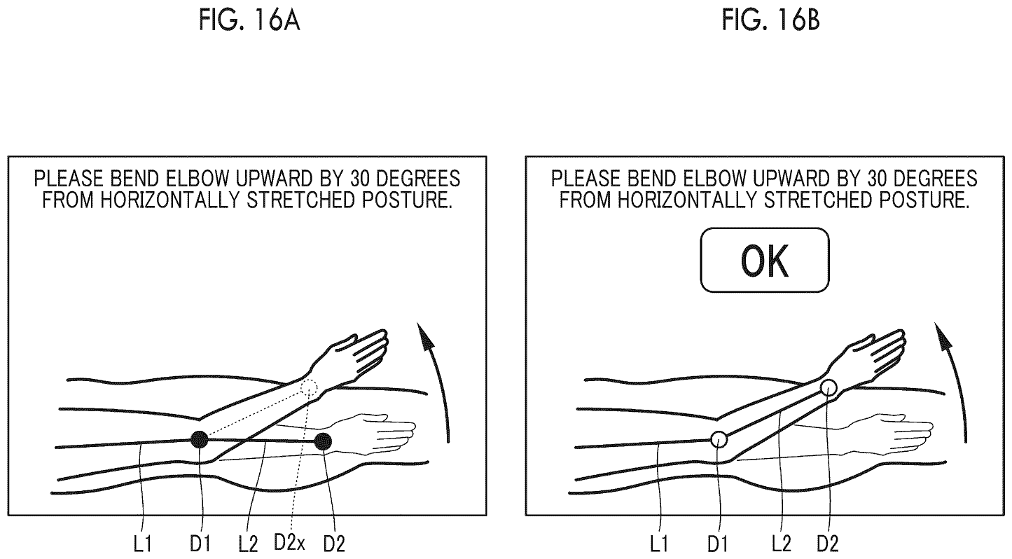

FIGS. 16A and 16B are diagrams showing an example of displaying the motion state of the subject. FIG. 16A shows an example of a display before the requested motion is performed, and FIG. 16B shows an example of a display in a case where the requested motion is performed.

In FIGS. 16A and 16B, a current motion state of the subject S is represented by using dots D1 and D2 and lines L1 and L2 in accordance with the skeletal model of the human body. Each of the dots D1 and D2 corresponds to the feature point detected by the motion detection unit 31F. In the example shown in FIGS. 16A and 16B, the dot D1 corresponds to the feature point detected as the right elbow. In addition, the dot D2 corresponds to the feature point detected as the right wrist. In addition, the line L1 as a link represents the right upper arm, and the line L2 represents the right forearm.

In FIG. 16A, the black dots D1 and D2 and the lines L1 and L2 indicated by solid lines indicate the current motion state of the subject S. In addition, a white dot D2x indicated by a broken line indicates a target position of the right wrist.

In a case where the subject S performs the requested motion, as shown in FIG. 16B, the colors of the dots D1 and D2 are changed. For example, the dots D1 and D2 are replaced with white dots. At the same time, letters “OK” are displayed.

The display control unit 31C3 controls the display based on the detection result of the feature point by the motion detection unit 31F. That is, each dot and line are displayed to correspond to the position of each detected feature point.

In this way, by displaying the information on the current motion state of the subject S together with the display information, it is possible to more appropriately instruct the subject S to perform the necessary motion. In addition, the subject can more easily understand the necessary motion. In particular, by displaying the target position together, the requested motion can be more easily understood. As a result, the examination can be smoothly executed.

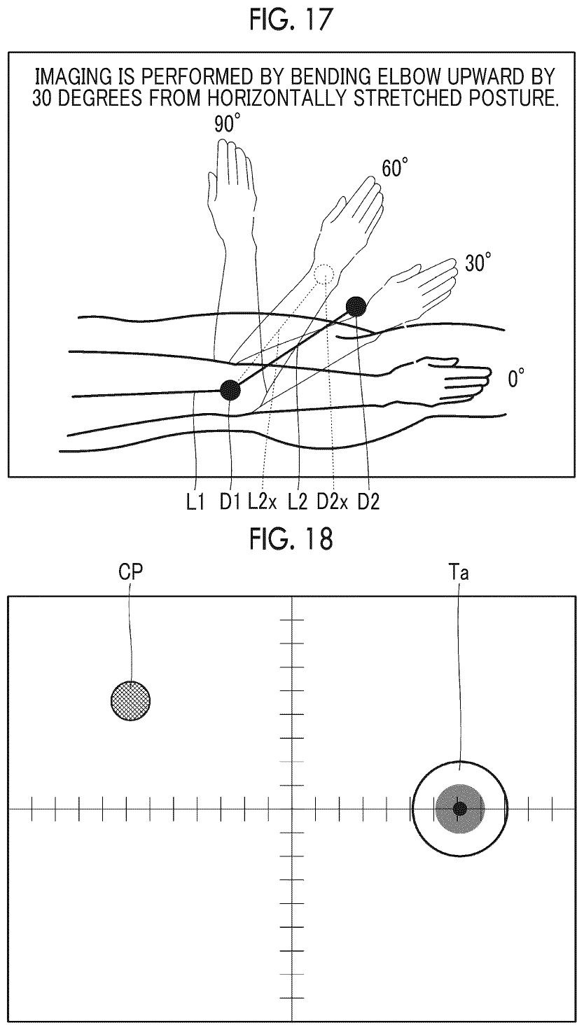

FIG. 17 is a diagram showing another example of displaying the motion state of the subject.

FIG. 17 shows an example of a case where the elbow is imaged a plurality of times by being bent in stages in the kinematic MRI examination of the elbow. In particular, FIG. 17 shows a case where the imaging is performed by bending the elbow upward by 30 degrees from the horizontally stretched posture. In this case as well, information indicating the current motion state of the subject S is displayed in addition to the information on the motion to be performed by the subject S. As in the modification example described above, in the present example as well, the motion state of the subject S is displayed by the dots D1 and D2 and the lines L1 and L2 based on the skeletal model of the human body.

FIG. 17 shows an example of a case where a next motion to be performed by the subject S is a motion of bending the elbow by 60 degrees. In this case, a dot D2x and a line L2x indicating the target position are displayed. In this way, in a case where the motion of moving a part of the body in stages is performed, the target position is displayed in sequence. As a result, it is possible to appropriately guide the subject S.

FIG. 18 is a diagram showing still another example of displaying the motion state of the subject. FIG. 18 shows an example of a case where the examination of the optic nerve is performed, and shows an example of a case where the subject S is asked to perform a motion of moving the viewpoint.

In this case as well, information indicating the current motion state of the subject S is displayed in addition to the information on the motion to be performed by the subject S. As described above, in a case where the subject S is asked to perform the motion of moving the viewpoint, the target Ta at which the subject S is gazed is displayed as the display information. In addition, as the information indicating the current motion state of the subject S, information indicating a current viewpoint position of the subject S is displayed. In FIG. 18, the current viewpoint position of the subject S is displayed by a circular mark CP. The subject S can easily grasp a position (position of the target Ta) to which the viewpoint is to be moved by checking the position of the mark CP.

In the case of the present example, the camera 14 images the eyes of the subject S. The motion detection unit 31F detects a position of the pupil from the image captured by the camera 14.

### (2) Guide

A guide (including presentation of an improvement point) for performing the requested motion may be provided to the subject S based on the detection result of the motion. In this case, the operation console 30 has a guide function.

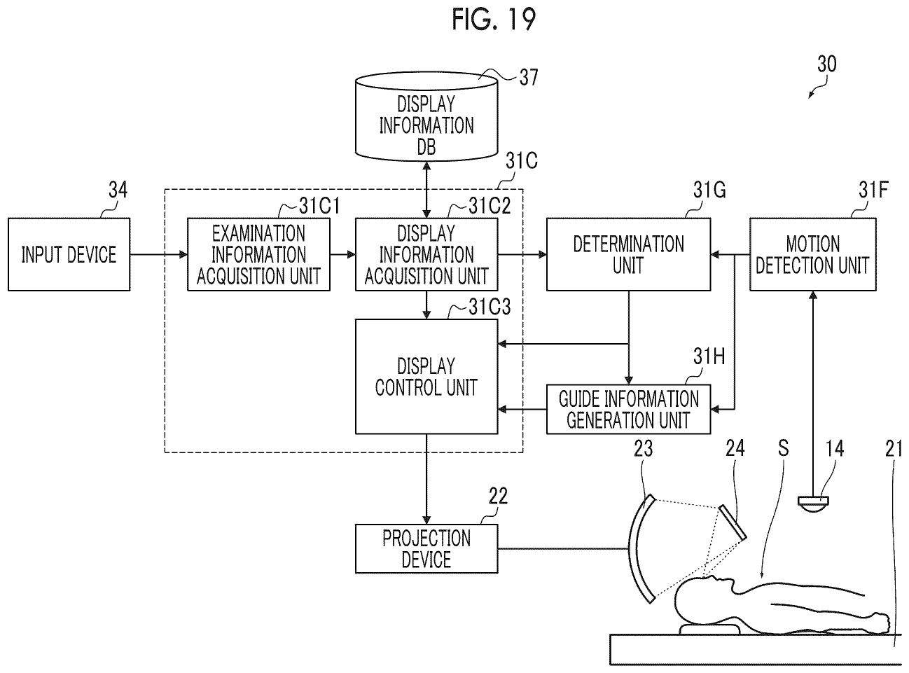

FIG. 19 is a block diagram of functions of the operation console regarding the guide function.

As shown in FIG. 19, the operation console 30 has a function of a guide information generation unit 31H that generates guide information. This function is realized by the processor 31 executing a predetermined program.

The guide information generation unit 31H determines whether or not it is necessary to generate the guide information based on the determination result of the determination unit 31G. That is, the guide information is generated in a case where the determination unit 31G determines that the requested motion has not been performed.

The guide information is information for the subject S to perform the requested motion, and is generated based on the current state of the subject S. The guide information generation unit 31H generates necessary guide information based on the detection result of the motion detection unit 31F. That is, information (information for improving the current state) for guiding the current state to a target state (position, posture, or the like) is generated. For example, in a case where the motion of bending the elbow upward by 30 degrees from the horizontally stretched posture is requested to the subject S, in a case where the detected motion of the subject S is a motion of bending the elbow upward by 20 degrees from the horizontally stretched posture, information for requesting to further bend the elbow upward by 10 degrees is generated as the guide information. In addition, for example, in a case where the motion of bending the elbow upward by 30 degrees from the horizontally stretched posture is requested to the subject S, in a case where the detected motion of the subject S is a motion of diagonally bending the elbow upward by 30 degrees from the horizontally stretched posture, information for requesting to correct the bending direction is generated as the guide information. In this way, the information on the requested motion is generated as the guide information based on the current state. The expression form is not particularly limited. For example, the guide information can be configured of text data in which a necessary motion is expressed in a sentence. In addition, it can be configured of data in which the necessary motion is expressed in an illustration or an animation, instead of the text data or in addition to the text data.

The display control unit 31C3 displays the guide information generated by the guide information generation unit 31H on the screen 23. As described above, as the guide information, information for guiding the subject S from the current state to the target state is displayed. For example, in the above example, a message such as “please bend elbow upward by additional 10 degrees” is displayed in a text at a predetermined position on the screen 23.

In this way, it is possible to easily ask the subject S to perform the requested motion by presenting the guide or the improvement point. In addition, the subject can easily execute the requested motion.

In the present example, the configuration is adopted in which the guide information is displayed on the screen 23, but a configuration may be adopted in which the guide information is output as a voice, in addition to the configuration in which the guide information is displayed on the screen 23 or instead of the configuration in which the guide information is displayed on the screen 23.

### (3) Notification of Determination Result or the Like

In the above-described embodiment, the configuration is adopted in which the determination result is displayed on the screen 23 and the subject S is notified of the determination result, but the method of notifying of the determination result is not limited to this. In addition, for example, a configuration may be adopted in which the subject is notified of the determination result by using a sound or a voice. The same applies to the guide information, and a configuration may be adopted in which the subject S is notified of the guide information by a voice.

### (4) Determination Method

Artificial intelligence (AI) can also be used to determine whether or not the requested motion has been performed. For example, a configuration can also be adopted in which an image captured by the camera 14 is added to the trained model to determine whether or not the requested motion has been performed. The trained model is generated through machine learning using predetermined learning data so that it can be determined from the image whether or not a predetermined motion has been performed.

### (5) Detection of Motion of Subject

In the above-described embodiment, the configuration is adopted in which the subject S under the examination is imaged with the camera 14 and the motion of the subject S is detected from the obtained image, but the method of detecting the motion of the subject S under the examination is not limited to this. For example, a configuration may be adopted in which the motion of the subject S is detected by using the image captured by the MRI apparatus 1.

### (6) Acquisition of Examination Information

In the above-described embodiment, the configuration is adopted in which the operator Op manually inputs the information on the examination to be executed (examination information) via the input device 34, but the method of acquiring the examination information is not limited to this. In general, in the MRI examination, information on the subject, information on the examination content, and the like are acquired (input) prior to the examination. Therefore, the examination information may be acquired by using these pieces of information. In addition, these pieces of information may be acquired from the outside (for example, an in-hospital system) through a network. Further, the information may be acquired from an imaging protocol.

### Third Embodiment

In the second embodiment, the configuration is adopted in which the motion of the subject S is detected, and the apparatus automatically determines whether or not the motion as requested has been performed based on the detection result. In the present embodiment, the detection result of the motion is presented to the operator Op, and the operator Op determines suitability of the motion.

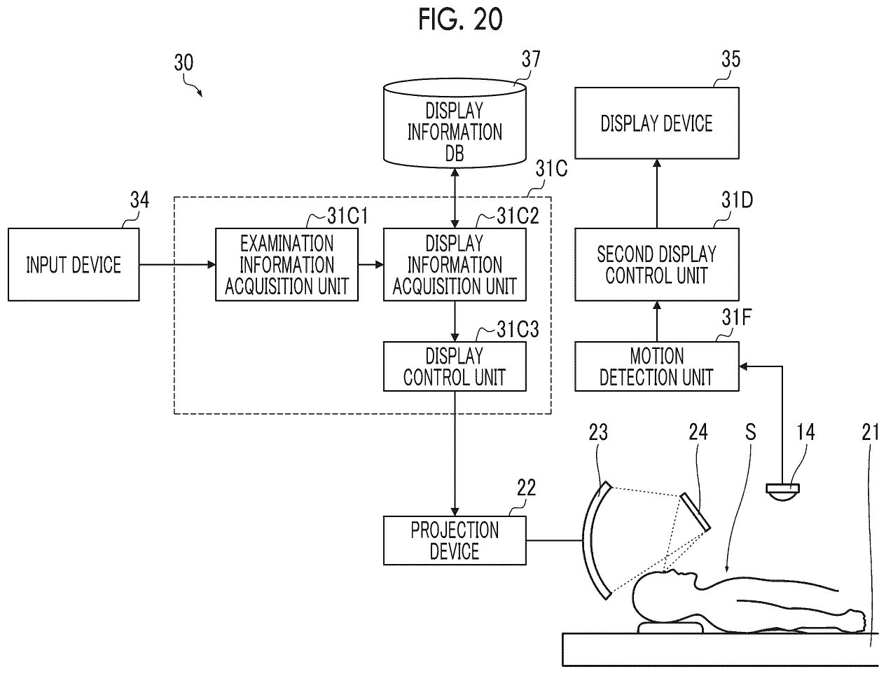

FIG. 20 is a block diagram of main functions of the operation console.

As shown in FIG. 20, in the MRI apparatus according to the present embodiment, the information on the motion of the subject S detected by the motion detection unit 31F is added to the second display control unit 31D. The second display control unit 31D displays the acquired information on the motion of the subject S on the display device 35. The display form is not particularly limited. For example, as in the above-described embodiment, the current motion state of the subject S can be displayed by using dots representing the feature points and lines connecting the dots (see FIGS. 16A and 16B). In this case, a target state (position, posture, or the like) may be displayed together. In the present embodiment, the display device 35 is an example of a second display unit that is visible to the operator Op.

As described above, in the MRI apparatus according to the present embodiment, the current motion state of the subject S is displayed on the display device 35. Therefore, in a case where the operator Op checks the display device 35, the operator Op can grasp the current motion state of the subject S. Therefore, the operator Op can determine whether or not the requested motion has been performed from the display of the display device 35. The operator Op notifies the subject of the determination result as necessary. For example, in a case where the requested motion has been performed, the fact that the requested motion has been performed is displayed (for example, “OK” is displayed) on the screen 23 via the projection device 22. In addition, in a case where the requested motion has not been performed, the information necessary for the requested motion is notified of. For example, information for guiding the position to the target position is notified of.

### Modification Example

### (2) Second Display Unit

In the above-described embodiment, the configuration is adopted in which the current motion state of the subject S is displayed on the display device 35 provided in the operation console 30, but the display destination of the current motion state of the subject S is not limited to this. A configuration may be adopted in which the current motion state is displayed on another display device located outside the imaging room 2. Therefore, for example, a configuration may be adopted in which the current motion state is displayed on a mobile terminal (for example, a tablet computer or a smartphone) comprising a display.

### (2) Presentation of Information to Both Subject and Operator

The detection result of the motion of the subject S may be presented to both the subject S and the operator Op.

In addition, a configuration may be adopted in which the apparatus automatically determines whether or not the motion as requested has been performed based on the detection result of the motion of the subject S, and notifies the operator Op of the determination result. For example, a configuration may be adopted in which the determination result is displayed on the display device 35 and the operator Op is notified of the determination result.

Further, a configuration may be adopted in which guide information for the subject S to perform the requested motion is generated based on the detection result of the motion of the subject S, and the operator Op is notified of the guide information.

### Fourth Embodiment

In general, in the MRI examination, an emergency call button is handed to the subject S. In a case where the subject S feels unwell or experiences any inconvenience during the examination, the subject S presses the button to notify the operator Op or the like of the feeling. However, in the examination in which a part of the body is moved, the button may hinder the examination. Therefore, in the present embodiment, an MRI apparatus in which the subject S under the examination can contact the operator Op or the like without using the emergency call button will be described.

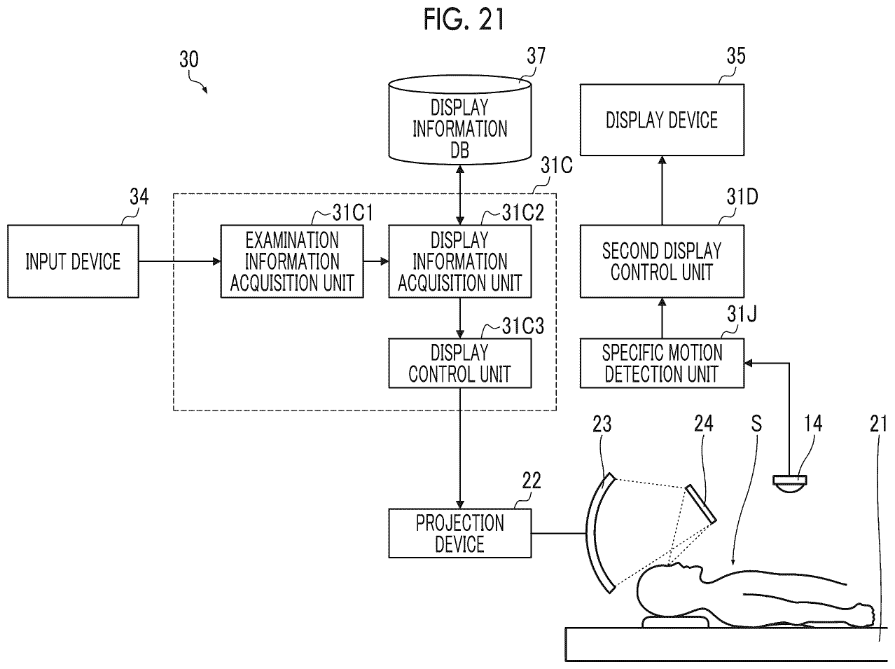

FIG. 21 is a block diagram of main functions of the operation console.

As shown in FIG. 21, the operation console 30 further has a function of a specific motion detection unit 31J. The specific motion detection unit 31J analyzes the image captured by the camera 14 to detect a specific motion by the subject S. Here, the specific motion includes the subject S taking a specific pose (posture) and making a specific sign. For each motion, the content of an intention to be communicated to the operator Op by the subject S or the like is determined.

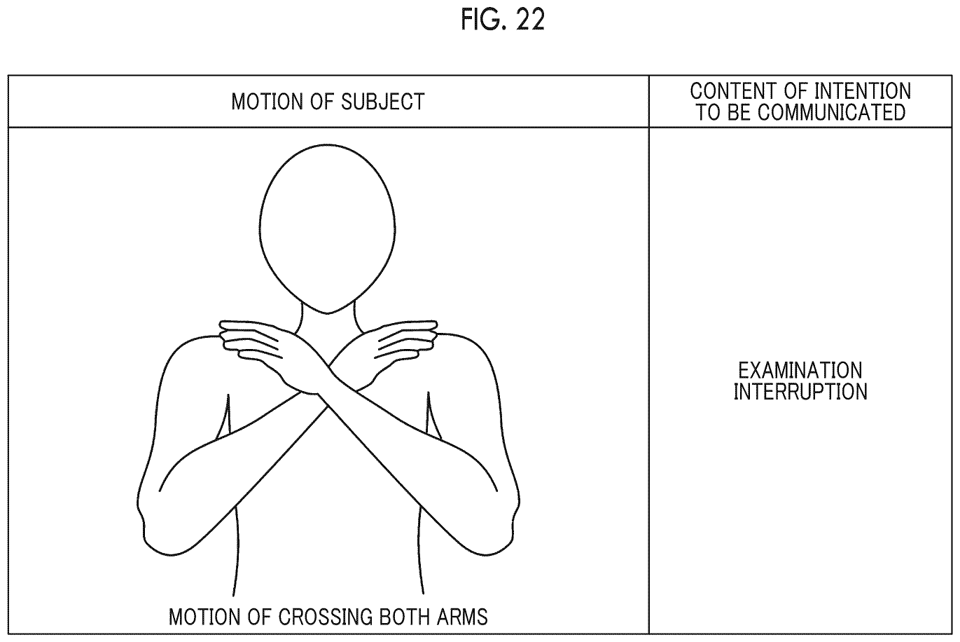

FIG. 22 is a diagram showing an example of a specific motion and a content of an intention to be communicated.

FIG. 22 shows an example of a case where an intention to interrupt the examination is communicated. In the example shown in FIG. 22, an intention to request the examination interruption is assigned to a motion (pose) of crossing both arms.

In addition, a negative (NO) intention can be assigned to a motion of shaking the head left and right, or a positive (YES) intention can be assigned to a motion of shaking the head up and down (nodding motion).

The subject S is notified of the detected motion and the content of the intention display assigned to each motion before the examination.

In a case where the specific motion detection unit 31J detects the specific motion from the image captured by the camera 14, the specific motion detection unit 31J outputs the information on the detected motion to the second display control unit 31D. For example, a trained model that has been trained through machine learning to detect the specific motion from the image can be used to detect the specific motion. In the present embodiment, the camera 14 and the specific motion detection unit 31J that detects the specific motion by the subject S from the image captured by the camera 14 are examples of a second detection unit.

The second display control unit 31D displays information corresponding to the motion detected by the specific motion detection unit 31J on the display device 35 and notifies the operator Op of the information. For example, in the example shown in FIG. 22, in a case where the specific motion detection unit 31J detects the motion with the intention to request the examination interruption (the motion of crossing both arms), the second display control unit 31D displays a message of “examination interruption” on the display device 35 and notifies the operator Op of the examination interruption.

As described above, with the MRI apparatus according to the present embodiment, in a case where the subject S performs a predetermined specific motion, information corresponding to the motion is displayed on the display device 35. As a result, the subject S under the examination can contact or communicate with the operator Op or the like without using the emergency call button.

### Modification Example

### (1) Notification Method

In the above-described embodiment, the configuration is adopted in which in a case where the specific motion is detected, the information corresponding to the detected motion is displayed on the display device 35 and the operator Op is notified of the information, but the method of notifying the operator Op of the information is not limited to this. Instead of the display on the display device 35 or in addition to the display on the display device 35, the information corresponding to the detected motion may be output as a voice or a sound to notify the operator Op of the information. In addition, a lamp may be turned on or off to notify the operator Op of the information.

### (5) Detection of Specific Motion

In the above-described embodiment, the configuration is adopted in which the subject S under the examination is imaged with the camera 14 and the specific motion is detected from the obtained image, but the method of detecting the specific motion is not limited to this. For example, a configuration may be adopted in which the specific motion is detected by using the image captured by the MRI apparatus 1.

### Other Embodiments

### (1) Processing Unit that Provides Each Function of Operation Console

A processing unit that provides each function of the operation console 30 can be configured by various processors. The various processors include, in addition to a graphic processing unit (GPU) which is a general-purpose processor, a programmable logic device (PLD) which is a processor whose circuit configuration can be changed after manufacturing such as a field programmable gate array (FPGA), and a dedicated circuitry which is a processor having a circuit configuration specifically designed to execute specific processing such as an application specific integrated circuit (ASIC). One processing unit may be configured of one of various processors or may be configured of two or more processors of the same type or different types. For example, one processing unit may be configured by a combination of a plurality of FPGAs or a combination of a CPU and an FPGA. In addition, a plurality of processing units may be configured of one processor. As an example of configuring a plurality of processing units with one processor, first, there is a form in which, as typified by computers used for a client, a server, or the like, one processor is configured by combining one or more CPUs and software, and the processor functions as a plurality of processing units. Second, as typified by a system on chip (SoC) or the like, a processor that realizes the functions of the entire system including the plurality of processing units by using one integrated circuit (IC) chip is used. As described above, various processing units are configured using one or more of the various processors as a hardware structure.

### (2) Medical Image Diagnostic Apparatus

In the above embodiment, a case where the present invention is applied to the MRI apparatus has been described as an example, but the application of the present invention is not limited to this. The present invention can be widely applied to a medical image diagnostic apparatus that captures a tomographic image of the subject, such as a CT apparatus.

### (3) Others

The above-described embodiments (including modification examples) can be appropriately combined.

### EXPLANATION OF REFERENCES

1: MRI apparatus
10: gantry
20: examination table
22: projection device
30: operation console
31: processor
31A: imaging control unit
31B: image acquisition unit
31C: first display control unit
31D: second display control unit
31E: recording control unit
33: auxiliary memory
34: input device
35: display device
37: display information database

What is claimed is:

1. A medical image diagnostic apparatus that images a subject in a bore, the apparatus comprising:
a first display unit that is visible to the subject under an examination; and
a processor,
wherein the processor
acquires first information including information on an examination target part of the subject, and
displays, on the first display unit, second information including information for causing the subject to move the examination target part based on the first information.

2. The medical image diagnostic apparatus according to claim 1,
wherein the information for causing the subject to move the examination target part is information on a motion of physically moving a specific part of a body.

3. The medical image diagnostic apparatus according to claim 2,
wherein the first information further includes information on an examination purpose and information on a motion to be performed by the subject.

4. The medical image diagnostic apparatus according to claim 3,
wherein the second information includes information on the part of the body to be moved and information on a method of moving the part.

5. The medical image diagnostic apparatus according to claim 4,
wherein the information on the method of moving the part includes information on a start position of the movement of the part and information on an end position of the movement of the part.

6. The medical image diagnostic apparatus according to claim 4,
wherein, in a case where the specific part of the body is moved in stages, the information on the method of moving the part includes information on a position at which the part is stopped.

7. The medical image diagnostic apparatus according to claim 3,
wherein the second information includes information representing the part of the body to be moved and a method of moving the part in an illustration or an animation.

8. The medical image diagnostic apparatus according to claim 2, further comprising:
a first detection unit that detects the motion of the subject in the bore,
wherein the processor
determines whether or not a requested motion has been performed based on a detection result of the first detection unit, and
notifies the subject of a determination result.

9. The medical image diagnostic apparatus according to claim 8,
wherein the processor displays, on the first display unit, information on the motion of the subject in the bore together with the second information based on the detection result of the first detection unit.

10. The medical image diagnostic apparatus according to claim 2, further comprising:
a first detection unit that detects the motion of the subject in the bore,
wherein the processor
generates guide information for the subject to perform a requested motion based on a detection result of the first detection unit, and
notifies the subject of the guide information.

11. The medical image diagnostic apparatus according to claim 10,
wherein the processor displays, on the first display unit, information on the motion of the subject in the bore together with the second information based on the detection result of the first detection unit.

12. The medical image diagnostic apparatus according to claim 2, further comprising:
a first detection unit that detects the motion of the subject in the bore; and
a second display unit that is visible to an operator,
wherein the processor displays, on the second display unit, information on the motion of the subject in the bore based on a detection result of the first detection unit.

13. The medical image diagnostic apparatus according to claim 12,
wherein the processor
determines whether or not a requested motion has been performed based on the detection result of the first detection unit, and
notifies the operator of a determination result.

14. The medical image diagnostic apparatus according to claim 12,
wherein the processor
generates guide information for the subject to perform a requested motion based on the detection result of the first detection unit, and
notifies the operator of the guide information.

15. The medical image diagnostic apparatus according to claim 1, further comprising:
a second detection unit that detects a specific motion by the subject,
wherein, in a case where the specific motion is detected by the second detection unit, the processor notifies an operator of the detected motion.

16. An operation method of a medical image diagnostic apparatus that images a subject in a bore, the method comprising:
acquiring first information including information on an examination target part of the subject; and
displaying, on a first display unit that is visible to the subject under an examination, second information including information for causing the subject to move the examination target part based on the first information.
